(19) United States
(12) Patent Application Publication
(10) Pub. No.: US 2011/0151484 A1
(43) Pub. Date: Jun. 23, 2011

(54) hERG MUTANTS AND USES THEREOF

(75) Inventors: James I. Vandenberg, New South Wales (AU); Mark J. Perrin, New South Wales (AU)

(73) Assignee: Victor Chang Cardiac Research Institute Limited, Sydney (AU)

(21) Appl. No.: 12/993,314

(22) PCT Filed: May 19, 2009
(86) PCT No.: PCT/AU2009/000624

§ 371 Date: Feb. 25, 2011

(30) Foreign Application Priority Data
May 20, 2008 (AU) 2008902492

(51) Int. Cl. G01N 33/53; C07H 21/00; C12N 15/63; C12N 5/10

(52) U.S. Cl. 435/7.21; 536/23.5; 435/320.1; 435/358; 435/369; 435/364

(57) ABSTRACT

An isolated nucleic acid molecule capable of expressing an human ether-à-go-go related gene (hERG) mutant that enhances hERG inactivation, wherein the mutation is not located in the vicinity of the drug-binding pocket within the cavity of the ion conduction pore of the hERG protein. Use of the isolated nucleic acid molecule in an assay for screening potential drug binding to hERG protein.

### TECHNICAL FIELD

The present invention relates to human ether-à-go-go related gene (hERG) mutants and uses thereof for screening potential drug binding to hERG protein.

### BACKGROUND ART

The human ether-à-go-go related gene (hERG) encodes the pore-forming subunit of the ion channel that conducts the rapid component of the delayed rectifier potassium current (I_{Kr}) in the heart. Gain and loss of function mutations in hERG may result in the clinical conditions of Short QT syndrome and Long QT syndrome respectively, underscoring the crucial role of hERG in maintaining electrical stability in the heart. The congenital form of LQTS is uncommon and may result from mutations in ten different genes—most encoding ion channels or their regulatory sub-units. Acquired LQTS, through drug-induced blockade of hERG, is more frequent and the most common reason for the withdrawal of medications from the pharmaceutical market.

Consequently, early assessment of a drug's affinity for hERG has been mandated, and obliges the need for a detailed understanding of how drugs bind to hERG.

It is well established that drugs bind in the central cavity of the pore region of hERG and that channels need to open before this can occur. At depolarized potentials, hERG channels can exist in either an open or an inactivated state, yet it has not been established whether the open or the inactivated state is the preferred drug binding state. Evidence in support of state-dependent drug binding primarily comes from studies showing reduced affinity for mutant channels that either abolish (S620T; G628C+S631C) or reduce inactivation (S631A). These mutations, however, lie proximate to the selectivity filter and putative drug-binding pocket (see FIG. 1) and so may affect drug-block directly or indirectly through local changes in the drug binding pocket. To address this concern the present inventors have investigated whether mutants to residues remote from the central pore that affect inactivation would affect drug binding to hERG in a manner similar to that reported for the S631A and S620T mutants.

The present inventors have now generated new mutants of hERG useful in screening for drug affinity for hERG.

### DISCLOSURE OF INVENTION

In a first aspect, the present invention provides an isolated nucleic acid molecule capable of expressing an human ether-à-go-go related gene (hERG) mutant that enhances hERG inactivation, wherein the mutation is not located in the vicinity of the drug-binding pocket within the cavity of the ion conduction pore of the hERG protein.

Preferably, the mutation is located on the alpha-helix of the S5P linker. More preferably, the mutation is N588E, H587K, G584S, or Q592D. It will be appreciated, however, that other mutations not located in the drug-binding pocket of the hERG protein, but which still enhance inactivation, would also be suitable for the present invention.

The present inventors have mutated residues N588, H587, G584 and Q592, located on the alpha-helix of the S5P linker, which is distant to the drug binding pocket, to either glutamate (N588E), lysine (N587K), serine (G584S) or aspartate (Q592D). Such mutations affect inactivation in hERG protein activity when expressed in a cell. Other candidate regions include all regions that are not part of the drug binding pocket. This includes the outer pore helix (e.g. F557A), voltage-sensor domain (e.g. A527V) both the N-terminal and C-terminal cytoplasmic domains, and the intracellular and extracellular linkers that join the transmembrane helical segments.

Preferably, the isolated nucleic acid molecule encoding an hERG mutant containing one or more mutations substantially as set out in FIG. 12 (SEQ ID NO: 1).

The nucleic acid molecule may contain one or more additional mutations such as L524W, L532P, V535A, A536V, K538Q that promote activation of the ion channel.

In a second aspect, the present invention provides vector for expressing an isolated nucleic acid molecule according to the first aspect of the present invention.

Preferably, the vector contains a promoter suitable for expression, preferably high level expression, in mammalian cells and an antibiotic resistance gene to aid selection of cells expressing the hERG channel.

In a third aspect, the present invention provides a stable cell line expressing an isolated nucleic acid molecule according to the first aspect of the present invention or a vector according to the second aspect of the present invention. The stable cell line expresses a hERG mutant protein.

Preferably, the cell is selected from, but not limited to, Chinese hamster ovary cells, human embryonic kidney cells or transformed African Green Monkey kidney fibroblast cells

Stable cell lines can be made using techniques known to the art and included antibiotic selection followed by clonal expansion.

The cell line is particularly suitable for screening potential drug binding to hERG protein.

In a fourth aspect, the present invention provides an assay for screening potential drug binding to hERG protein comprising:

a stable cell line according to the third aspect of the present invention; and

means to determine activity of the hERG protein in the cell.

The assay may involve either ion flux or voltage changes then using patch clamp assays, Rb+ efflux assays as a surrogate for K+ flux or fluorescence to assay voltage changes. Drugs will be incubated with cells and the effects on ion flux or voltage monitored. Assay conditions may include presence of high Rb+ or high K+ (to enhance entry into inactivated state)—then ion flux or voltage assayed following removal of the high K+ or Rb+. In some circumstances, the incorporation of mutations that enhance activation of hERG channels may obviate the need for high Rb+ or high K+ conditions.

In a fifth aspect, the present invention provides use of the isolated nucleic acid molecule according to the first aspect of the present invention or use of the cell line according to the third aspect of the present invention in an assay for screening potential drug binding to hERG protein.

The present inventors have characterised the binding of four high-affinity blockers (dofetilide, cisapride, astemizole, and terfenadine) as well as four low-affinity blockers (quinidine, perhexiline, erythromycin and dl-sotalol). All four high affinity blockers exhibited reduced affinity for the inactivation deficient mutants (N588K and S620T) whereas only dl-sotalol among the low affinity blockers showed reduced affinity for N588K. In all cases where binding was affected by inactivation deficient mutants, the affinity for S620T was markedly lower than for N588K mutant channels. A kinetic model of drug binding indicated that the difference between drug binding to wild type (WT), N588K and S620T channels can be explained by the kinetics of drug binding with the affinity for the open state being reduced 4-70 fold compared to the inactive state depending on the particular drug studied.

The data strongly suggests state dependent binding is necessary, but not sufficient for high affinity blockade of hERG. Further, the affinity of drugs for the S620T mutant represent their true affinity for the open conformation of the channel and the measured affinity for the WT channel is a weighted average of the affinity for the open and inactivated states. A corollary of our findings is that high throughput screens that do not assay inactive state binding may grossly underestimate the true affinity for WT-hERG channels.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this specification.

In order that the present invention may be more clearly understood, preferred embodiments will be described with reference to the following drawings and examples.

### BRIEF DESCRIPTION OF THE DRAWINGS

FIG. 1: Schematic diagram of the S5, S6, pore helix, and S5P linker regions of two hERG subunits. The most important molecular determinants of drug-block are denoted with grey circles. Commonly employed mutants that disrupt inactivation are displayed with white stars. Note that G648 is probably not exposed to the pore cavity, and is thought to influence drug block by determining the conformation of the drug-binding pocket; V659 may influence drug-block by altering the gating characteristics of the channel. The S5P linker is dashed, as its exact position relative to the pore is unknown.

FIG. 2: Conductance voltage curves for N588E-hERG (◯), WT-hERG (▪), and N588K-hERG (). Data points are mean ±SEM and the curve fitted to the data is a Boltzmann function with a V_{0.5} of inactivation of −114±3 mV and slope factor of −19 mV (n=13) for N588E-hERG, V_{0.5} of −78±2 mV and slope factor of −26 mV (n=11) for WT-hERG, and a V_{0.5} of 45±4 mV and slope factor of −14 mV (n=6) for N588K-hERG.

FIG. 3: Typical examples of current traces recorded from the drug-block voltage protocol illustrated at top. A. WT-hERG, B. N588E-hERG, and C. N588K-hERG, in the presence of 30 nM cisapride. Dashed lines represent zero current in each case. Note the different scales on each recording; A. and B. are currents recorded during the revelatory voltage step, while C. displays the non-rectifying current of the 3s depolarising step in N588K-hERG. The percentage of drug-block for each recording was determined by dividing the current measured at the end of the 3s depolarising step to +20 mV after application of the drug (I_{drug}) by the current measured at the beginning of the recording (I_{control}). In the case of WT-hERG and N588E-hERG, the current at this point was determined by fitting an exponential curve to the first part of the deactivating current during the revelatory voltage step and extrapolating this back to the end of the depolarising step, as illustrated in A. and B.

FIG. 4: Hill Plots of high-affinity hERG blockers. Drug binding to WT-hERG (▪), N588E-hERG (◯), and N588K-hERG () for A. astemizole, B. cisapride, C. dofetilide D. terfenadine. Each data point represents I_{[drug]}/I_{control}±S.E.M for n=4-9 cells. E. Summary data showing the mean IC₅₀±S.E.M for each drug. Significant differences between WT-hERG and N588K-hERG are denoted by *. All values are shown in Table 1.

FIG. 5: Typical examples of current traces recorded from A. WT-hERG, B. N588E-hERG, and C. N588K-hERG in the presence and absence of 3 μM quinidine. The drug-block voltage protocol is shown at top. Dashed lines represent zero current in each case. Note the >50% of block of N588K-hERG by 3 μM quinidine which is similar to the percentage block in WT-hERG and N588E-hERG. Rates of deactivation in N588E-hERG are faster than WT-hERG due to the use of a −120 mV ‘revelatory’ voltage step, in order to produce adequate opening of channels to measure current. The rate of deactivation appears to slow in A. and B. with application of drug; this is probably due to some drug unblocking during this step, rather than a true effect on the kinetics of deactivation.

FIG. 6: Hill plots of low affinity hERG blockers. Drug binding to WT-hERG (▪), N588E-hERG (◯), and N588K-hERG () for A. quinidine B. perhexiline C. erythromycin D. dl-sotalol. Each data point represents I_{[drug]}/I_{control}±SEM for n=4-6. E. Summary data showing the mean IC₅ ±S.E.M for each drug. Significant differences between WT-hERG and N588K-hERG are denoted by *. All values are shown in Table 1.

FIG. 7: A. Conductance voltage curves for S631A-hERG(Δ), and S620T-hERG(□). Data points are mean ±SEM and the curve fitted to the data points is a Boltzmann function. The V_{0.5} of steady state inactivation for the S631A-hERG mutant is 43±2 mV with a slope factor of −16.5 mV (n=6). S620T-hERG does not inactivate in the voltage range shown. Dotted, plain, and dashed lines show data for N588E-hERG, WT-hERG, and N588K-hERG, reproduced from FIG. 2. B. The affinity of dofetilide for S631A-hERG (Δ), and S620T-hERG (□). Dotted, plain, and dashed lines show data for N588E-hERG, WT-hERG, and N588K-hERG respectively, reproduced from FIG. 4C. Note the similar drug affinity for the N588K and S631A cell constructs, and the marked right shift (reduced affinity) of dofetilide for the S620T construct.

FIG. 8. Markov model of drug-binding to hERG. Cₓ=closed states. O=open state. I=inactivated state. OD=drug bound to open state. ID=drug bound to inactivated state.

FIG. 9: Dofetilide Hill curves with data points for N588K-hERG and the modelling curve superimposed. Thin plain line, dotted line, and dashed line show WT-, N588K-, and S620T-hERG respectively. The modelled line is thick black. Experimental IC₅₀ values for dofetilide block of N588K, and the modelled values respectively: 377±57.8 vs 302 nM.

FIG. 10: Hill curves for A. astemizole B. cisapride C. terfenadine and D. dl-sotalol with data points for N588K-hERG and the modelling curves superimposed. Thin plain line, dotted line, and dashed line show WT-, N588K-, and S620T-hERG respectively. Modelled lines are thick black. Experimental IC₅₀ values for drug block of N588K and the modelled values respectively: astemizole—14.8±1.1 vs 13.6 nM; cisapride—55.9±4.2 vs 77.3 nM; terfenadine—170±32.3 vs 177 nM; dl-sotalol 1392±266.5 vs 1461 μM. IC₅₀ values for S620T are presented in Table 2.

FIG. 11: Quinidine current clamp assay. Typical modelling experiments (a.), and raw data (b.), for quinidine current clamping experiments. In b. drug is applied at the beginning of the tracing, and results in depolarisation of the cell membrane. The magnitude of voltage change may be converted to a percentage drug block, and plotted on a standard Hill curve (c.) By this means the IC50 of drug block in the current clamp assay may be compared to the IC50 of drug block in the voltage clamp assay.

FIG. 12: Synthetic hERG cDNA (1..3486). Contains possible mutations for enhancement of inactivation (G584S, H587K, N588E) and activation (L524W, L532P, V535A, A536V, K538Q. These mutations are underlined and the variable bases are indicated using the IUPAC codes. In addition there are multiple silent mutations introducing restriction sites.

In summary,

L524W (YKN)
(SEQ ID NO 2)
CTT, CTC, CTA, CTG, TTA, TTG, or TGG
L532P (YYN)
(SEQ ID NO 3)
CTT, CTC, CTA, CTG, TTA, TTG, CCT, CCC, CCA, or
CCG
V535A (GYN)
(SEQ ID NO 4)
GTT, GTC, GTA, GTG, GCT, GCC, GCA, or GCG
A536V (GYN)
(SEQ ID NO 5)
GTT, GTC, GTA, GTG, GCT, GCC, GCA, or GCG
K538Q (MAR)
(SEQ ID NO 6)
AAA, AAG, CAA, or CAG
G584S (DSN)
(SEQ ID NO 7)
GGT, GGC, GGA, GGG, TCT, TCC, TCA, TCG, AGT, or
AGC
H588K (MAN)
(SEQ ID NO 8)
CAT, CAC, AAA, or AAG
N588E (RAY)
(SEQ ID NO 9)
AAT, AAC, GAT, or GAC

IUPAC codes for nucleotides in sequence:

A Adenine

C Cytosine

G Guanine

T Thymine

U Uracil

R Purine (A or G)

Y Pyrimidine (C, T, or U)

M C or A

K T, U, or G

W T, U, or A

S C or G

B C, T, U, or G (not A)

D A, T, U, or G (not C)

H A, T, U, or C (not G)

V A, C, or G (not T, not U)

N Any base (A, C, G, T, or U)

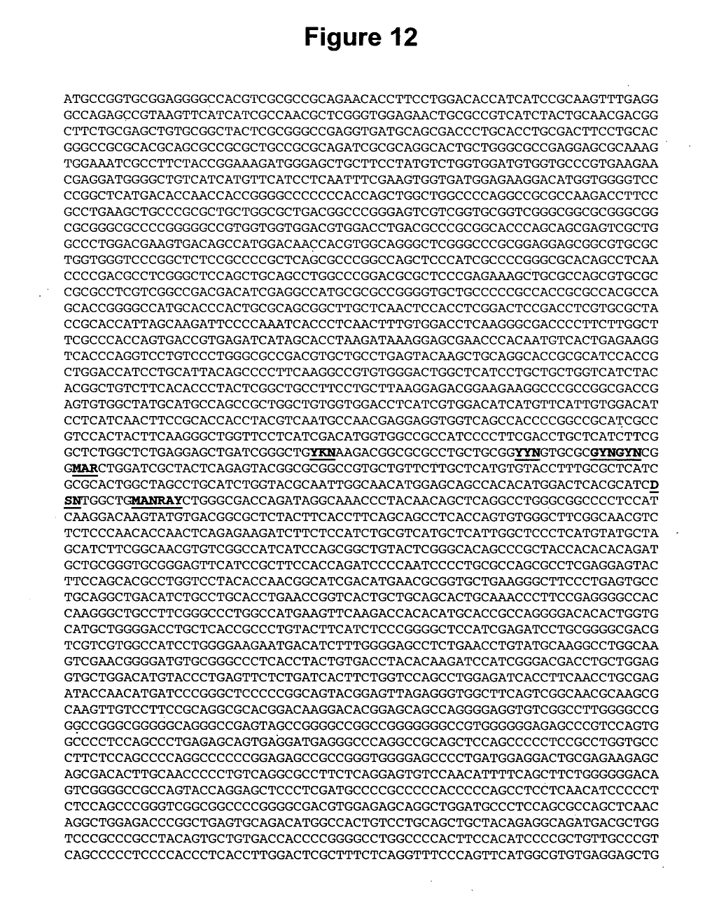

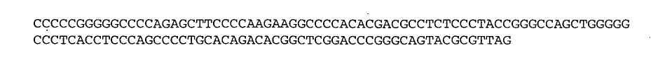

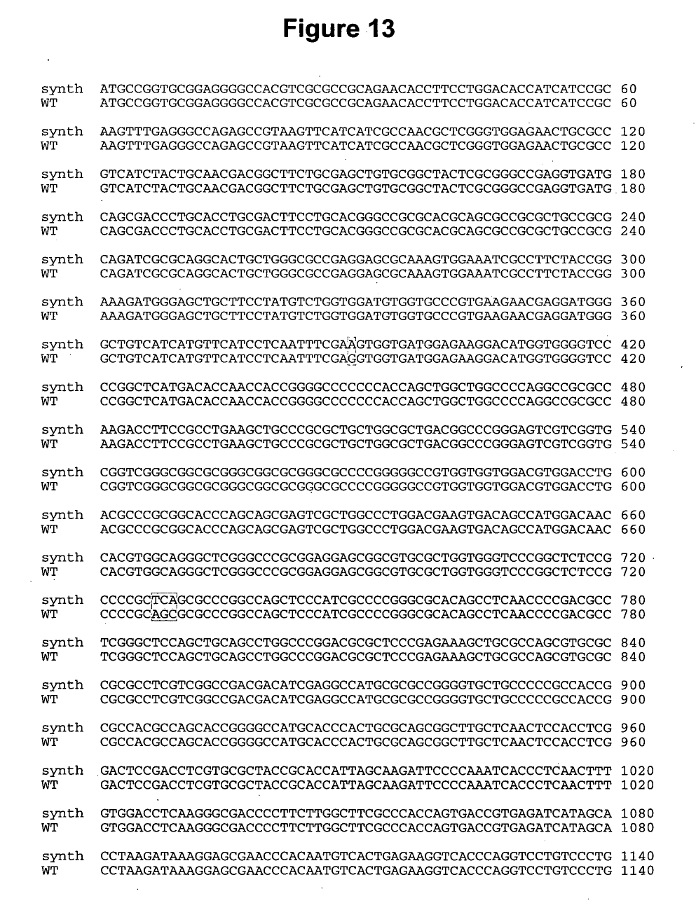

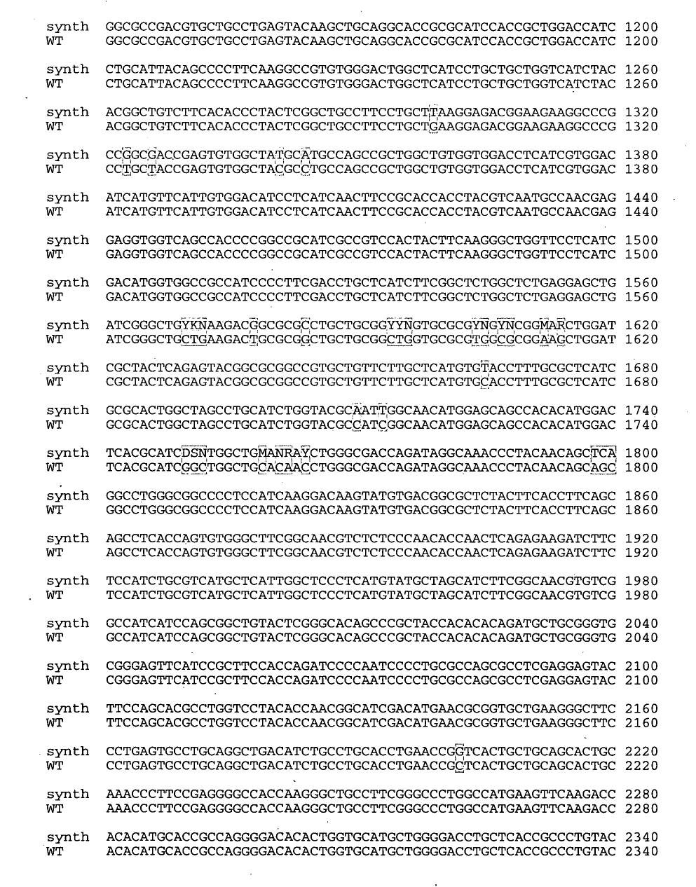

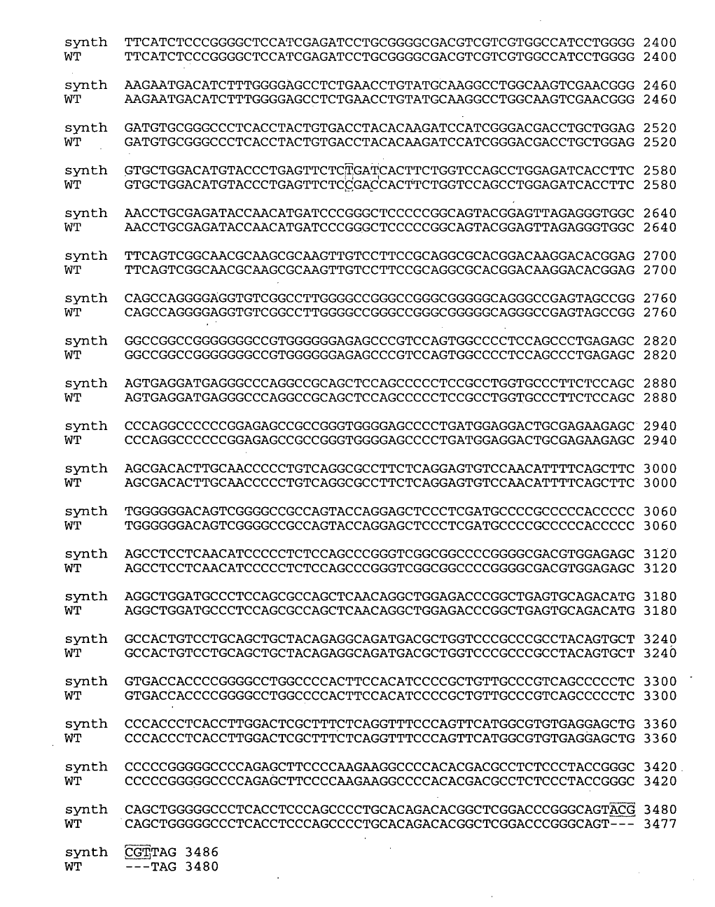

FIG. 13: Comparison of nucleotide sequence of synthetic construct) shown in FIG. 12 (SEQ ID NO 1) to wild type hERG cDNA (SEQ ID NO 10).

### MODE(S) FOR CARRYING OUT THE INVENTION

The present invention is described herein using several definitions, as set forth below and throughout the specification.

As used herein, unless otherwise stated, the singular forms “a,” “an,” and “the” include plural reference. Thus, for example, a reference to “an oligonucleotide” includes a plurality of oligonucleotide molecules, and a reference to “a nucleic acid” is a reference to one or more nucleic acids.

As used herein, “about” means plus or minus 10%.

### METHODS

### Molecular Biology

Experiments on wild type (WT) hERG channels were performed using a Chinese hamster ovary (CHO) cell line stably expressing the hERG K⁺channel constructed as previously described (Walker BD et al., Br J Pharmacol 1999;128(2):444-450). CHO cells were cultured in D-ME/F12 with 10% FBS and maintained at 37° C. in 5% CO₂. Cells were studied at least 24 h after being plated on microscope coverslips. Mutant hERG constructs were generated using the Megaprimer PCR method. Mutant constructs (N588K, N588E, S631A or S620T) were sequenced to ensure that only the correct mutation had been made and then subcloned into the pIRES2-eGFP vector. Blank CHO cells were plated onto sterilised glass coverslips. After 24 h, the cells were transfected with mutant hERG constructs using PolyFect Transfection Reagent (Qiagen) according to the manufacturer's instructions. After transfection, the cells were incubated at 37° C. for a minimum of 1 day prior to electrophysiological study. Successfully transfected cells, identified by the expression of eGFP, were studied using the whole-cell configuration of the patch clamp technique.

### Electrophysiology

Borosilicate glass tubing patch pipettes, with resistances of 1-4 MΩ when filled with internal solution, were made using a vertical 2-stage puller (PP-830; Narishige, Japan). The internal solution contained (in mM) 120 potassium gluconate, 20 KCl, 1.5 MgATP, 5 EGTA and 10 HEPES (pH 7.3 with KOH). Membrane potentials were adjusted by −15 mV to correct for the junction potential (Barry, 1994) between low Cl⁻pipette solution and external bath solution (see below). Currents were amplified (Axopatch 200B amplifier; Molecular Devices, Sunnyvale, Calif.) and digitised (DigiData 1200; Molecular Devices) before storage on a personal computer. Capacitance current transients were electronically subtracted, and series resistance compensation was ˜80% for all experiments. Current signals were digitised at 5 kHz and low-pass filtered at 2 KHz. Some current traces were leak-subtracted off-line. Acquisition was performed using pClamp software.

### Solutions and Drugs

Cells were superfused with a Tyrode solution containing (in mM): 130 NaCl, 5 KCl, 1 MgCl, 1 CaCl₂, 12.5 Glucose, 10 Hepes. Cisapride, Astemizole, Terfenadine, Erythromycin, Dofetilide, and Quinidine, were prepared as stock solutions in dimethyl sulphoxide (DMSO) and subsequently diluted as required with superfusate (maximum final DMSO concentration=0.1% vv⁻¹). Dl-Sotalol was prepared as a stock solution in Tyrode solution, and Perhexiline as a stock solution in methanol. We have previously found that DMSO at 0.1% vv⁻¹ has no effect on the parameters under study.

### Voltage Protocols

### Steady State Inactivation

From a holding potential of −80 mV the membrane voltage was stepped to +40 mV for 3 seconds to fully activate the channels. For cells expressing WT-hERG and N588E-hERG, the second pulse consisted of a voltage step from +40 mV to −160 mV in 10 mV decrements. For cells expressing N588K, a range of +120 mV to −20 mV was employed. For voltage steps to <−30 mV, an exponential curve was fitted to the first phase of deactivation and extrapolated back to the beginning of the second pulse at which point its magnitude represents the current that the channel would have passed if recovery from inactivation were instantaneous. Channel conductance was plotted against voltage to yield the voltage-dependent steady state inactivation curve (properly, the steady state recovery from inactivation) and the data fitted with a Boltzmann function:

$$\frac{G}{{G}_{max}} = \frac{1}{( 1 + {e}^{\frac{{V}_{0.5} - {V}_{t}}{k}} )}$$

V_{0.5} —half inactivation voltage

k—slope factor

### Drug Block

As this was a comparative study, care was taken to ensure similar conditions for drug testing in WT and mutant channels. However, due to the unique gating characteristics of N588E-hERG and N588K-hERG, slight alterations in the voltage protocol and method of measurement were used. Currents were measured with a two-step voltage protocol; a first step to +20 mV for 3 seconds to fully activate the channels and a second step to a negative membrane potential, usually −110 mV (but see below for variations). During this second voltage step hERG channels quickly recover from inactivation and proceed to deactivate. This second step was termed ‘revelatory’, because it allowed us to estimate the total conductance of activated channels (open and inactive) after the 3s period of depolarisation.

The revelatory step was typically recorded at −110 mV, although in some N588E-hERG cells a voltage step to −120 mV was employed to allow adequate recovery from inactivation for current measurement. Conversely, a less negative voltage was used for a few N588K-hERG cells in order to minimise series resistance errors due to the large activating current in this non-rectifying construct. Drug block was calculated as I_{[drug]/I}_{control}, with all currents measured at the end of the activating step. For N588E-hERG and WT-hERG a single exponential fit was applied to the first part of the current trace during the revelatory step and extrapolated back to the end of the activating step. In this way current was measured at the same time point for all cells.

Voltage-protocols were repeated at 0.1 Hz. Control currents were recorded 3-5 minutes after patch rupture, i.e. the time take for current levels to stabilise at a steady state level. The first drug was applied, with solution exchange typically taking less than 10s. Recording continued until a new steady state block was reached (typically 2-4 minutes). Between 2 and 4 doses of drug were applied to each cell with most experiments completed within 20 minutes.

### Data Analysis

Initial data analysis was performed using the Clampfit module of the pClamp 9.0 software. Subsequent data analysis and preparation of data for figures were performed with Mathematica 6 (Wolfram Technologies). All data are expressed as mean ±SEM (n) and statistical significance (P<0.05) was determined using paired t-tests.

### Vectors

hERG cDNA was cloned into a mammalian expression construct containing a CMV viral promoter to direct expression and a G418 antibiotic selection cassette to permit selection of stable cell lines. Stable cell lines were generated as previously described (Walker BD et al., Br J Pharmacol 1999;128(2):444-450). It will be appreciated that any suitable vector known to the art would be suitable.

### Host Cells

The preferred host cell is Chinese hamster ovary cells, human embryonic kidney cells or transformed African Green Monkey kidney fibroblast cells (COS7). Techniques for obtaining stable host cells expressing a mutant protein are well known to the art and can be found from online protocols under the Biology-Transfection-Stable-Transfection online document used by persons in this art.

### Screening Assays

The mutant hERG constructs and/or stable cell lines can be assayed using conventional patch clamp electrophysiology assays (Perrin MJ et al, Mol Pharmacol. 2008 November; 74(5):1443-52), Current clamp assays from moleculardevices, Rb+ flux assays (Rezazadeh S et al., J Biomol Screen. 2004 October;9(7):588-97) or voltage following assays utilizing voltage sensitive dyes (Dorn A et al, J Biomol Screen. 2005 June; 10(4):339-47).

### RESULTS

V₀.₅ of Steady State Inactivation in WT-hERG, N588E-hERG and N588K hERG Expressed in CHO Cells

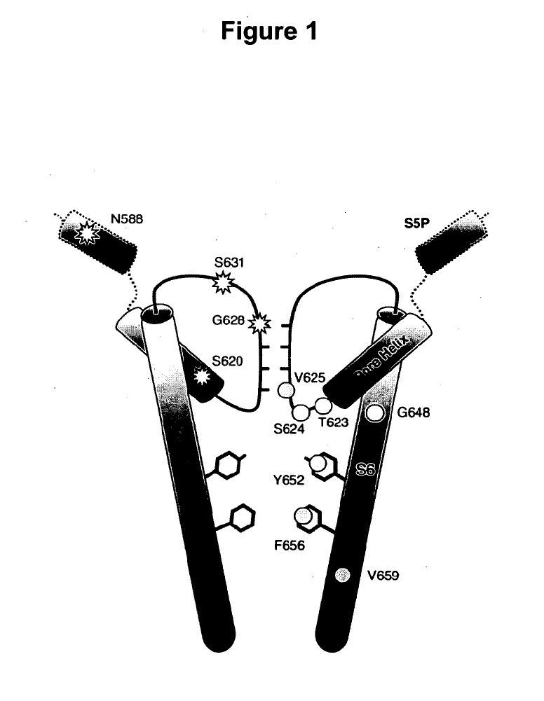

To investigate the link between drug binding and state dependence we chose to use mutants of residue N588 located in the α-helix of the S5P linker of hERG (FIG. 1). This residue has two important features, firstly, it is though to be located distant to the drug binding pocket and secondly it is possible to titrate the voltage-dependence of inactivation of the channels by introducing different charges at this residue.

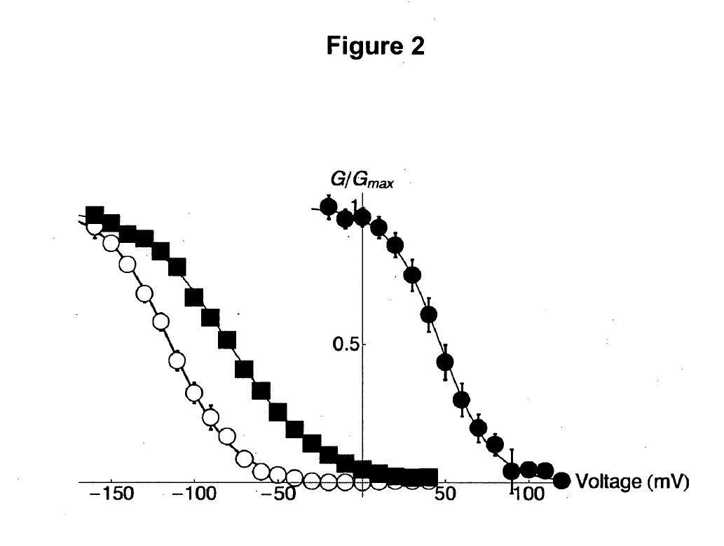

Studies of N588 mutant channels have been carried out using the Xenopus laevis oocyte expression system; however, mammalian cells are the preferred heterologous expression system to use for drug binding studies. Therefore, we first wanted to confirm that the properties of N588 mutant channels were similar in mammalian cells and Xenopus laevis oocytes. When expressed in CHO cells, the V_{0.5} of inactivation for N588E-hERG was −114 mV±3 mV with a slope factor of −19 mV±1 mV, (n=13); the V_{0.5} of inactivation for WT-hERG was −78 mV±2 mV with a slope factor of −27 mV±2 mV, (n=11); and the V_{0.5} of inactivation for N588K-hERG was 45 mV±4 mV with a slope factor of −14 mV±1 mV, (n=6) (FIG. 2). All values are similar to those reported for Xenopus oocytes. The reversal potential for N588K-hERG; −83.4±2.0 mV (n=5), was slightly shifted compared to that for WT-hERG; −88.5±2.2 mV, n=10. The N588K-hERG channels are nevertheless still highly selective for K⁺over Na⁺. The reversal potential in the N588E-hERG construct could not be determined due to the small magnitude of its activating currents. However, when expressed in oocytes with much larger currents it was found to be similar to WT-hERG. These properties together make N588 mutant constructs ideal for the investigation of inactivation mediated drug-binding to hERG.

High Affinity Drug Binding is modulated by N588 Charge Mutants

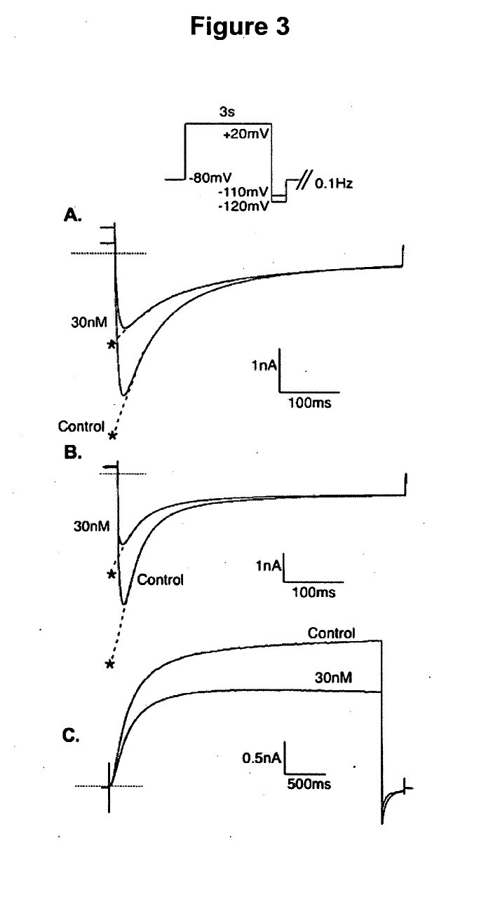

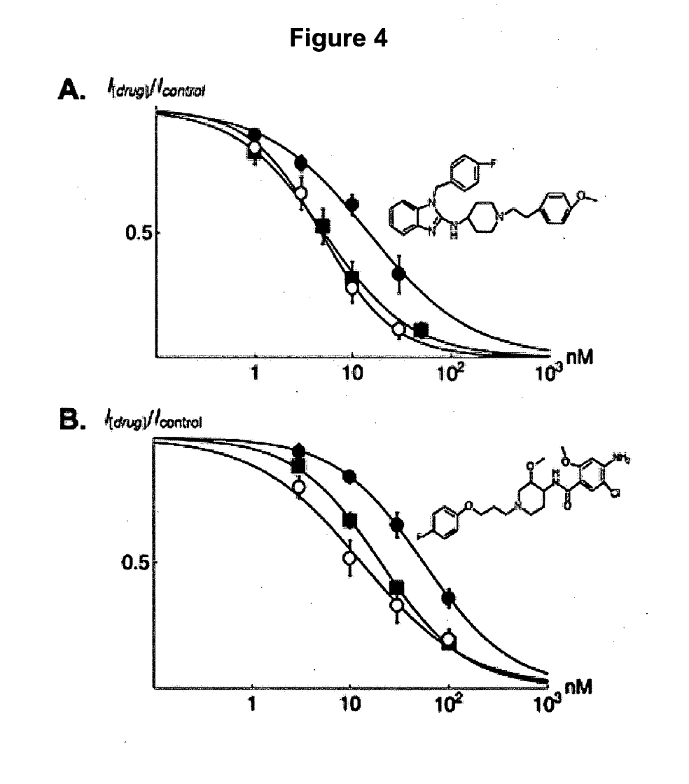

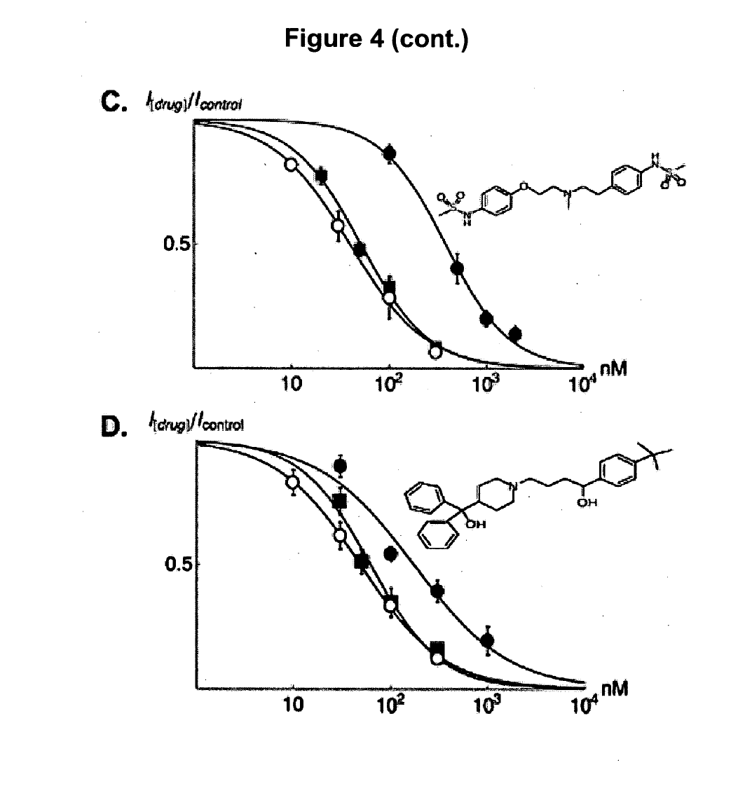

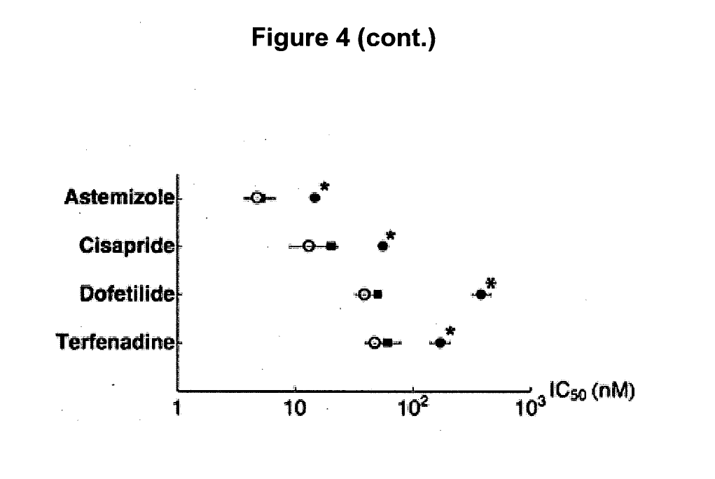

We initially investigated the affinity of 4 drugs, previously established to block hERG in the low nanomolar range—astemizole, cisapride, dofetilide, and terfenadine, for N588E-hERG, WT-hERG and N588K-hERG expressed in CHO cells. FIG. 3 shows typical examples of WT-, N588E- and N588K-hERG traces under control conditions and after 5 minutes equilibration with 30 nM cisapride. Percentage of drug-block was measured at the end of the 3s-activating step to +20 mV in all cells. This was performed directly in N588K (FIG. 3c), or else by fitting a single exponential curve to the first part of the current trace during the revelatory step in N588E-hERG (FIG. 3b) and WT-hERG (FIG. 3a) and extrapolating this back to the end of the activating step. The data in FIG. 3 indicate that 30 nM cisapride caused less block of N588K-hERG channels compared to N588E- or WT-hERG. This is more clearly seen from the summary Hill-plots shown in FIG. 4; cisapride affinity for WT=hERG: 20.5±2.2 nM (n=4) was similar to that for N588E-hERG: 13.1±4.9 nM (n=4) but much reduced for N588K-hERG: 55.9±4.2 nM (n=4). All four high-affinity blockers showed a similar pattern; reduced affinity for N588K-hERG compared to WT- and N588E-hERG (see FIG. 4). The affinities for all drugs for WT and N588 mutant constructs are summarised in Table 1.

TABLE 1
Affinities for all drugs for WT and N588 mutant constructs

| Drug | N588E | WT-hERG | N588K |
|---|---|---|---|
| Astemizole | 4.8 ± 1.2 nM | 5.1 ± 1.4 nM | 14.8 ± 1.1 nM |
| | (5); | (6); | (5)⁺*; |
| | h = 1.2 | h = 1.0 | h = 0.8 |
| Cisapride | 13.9 ± 4.9 nM | 20.5 ± 2.2 nM | 55.9 ± 4.2 nM |
| | (4); | (6); | (6)⁺*; |
| | h = 0.8 | h = 1.0 | h = 0.9 |
| Dofetilide | 39.4 ± 7.3 nM | 51.0 ± 1.3 nM | 377.3 ± 57.8 nM |
| | (5); | (9); | (5)⁺*; |
| | h = 1.1 | h = 1.3 | h = 1.3 |
| Terfen- | 48.2 ± 10.6 nM | 61.4 ± 16.2 nM | 170.2 ± 32.3 nM |
| adine | (5); | (9); | (7)⁺*; |
| | h = 1.0 | h = 1.2 | h = 0.9 |
| Quinidine | 3.71 ± 0.4 μM | 3.2 ± 0.3 μM | 3.9 ± 0.3 μM |
| | (4); | (5); | (4); |
| | h = 1.1 | h = 0.8 | h = 0.9 |
| Perhexiline | 5.0 ± 1.8 μM | 5.9 ± 0.9 μM | 4.7 ± 1.7 μM |
| | (5); | (7); | (5); |
| | h = 0.8 | h = 1.1 | h = 1.2 |
| Erythro- | 98.4 ± 39.7 μM | 129.5 ± 59.9 μM | 151.1 ± 49.4 μM |
| mycin | (5); | (4); | (4); |
| | h = 0.8 | h = 0.7 | h = 0.8 |
| dl-Sotalol | 585.6 ± 178.6 μM | 515.5 ± 35.5 μM | 1392.0 ± 266.5 μM |
| | (4); | (4); | (4)⁺*; |
| | h = 0.8 | h = 0.9 | h = 0.9 |

### Low Affinity Drug Binding to N588 Charge Mutants

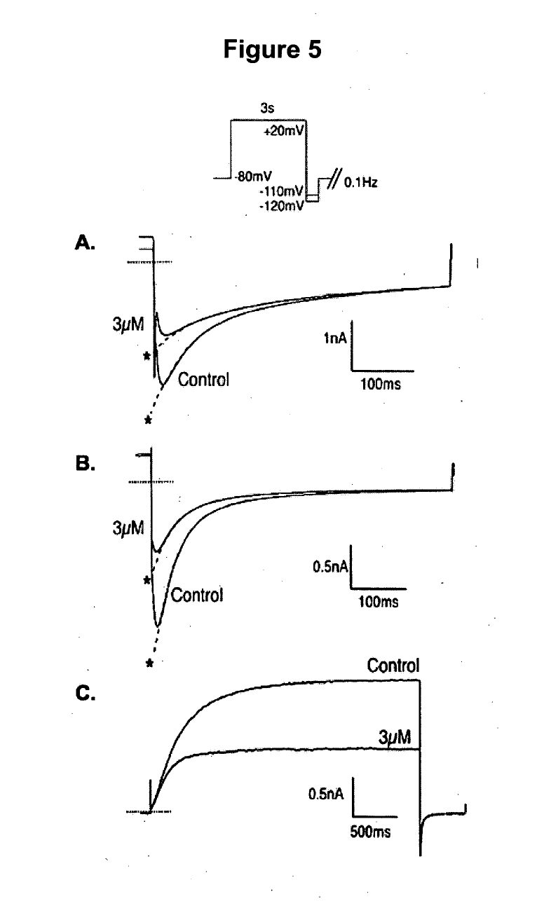

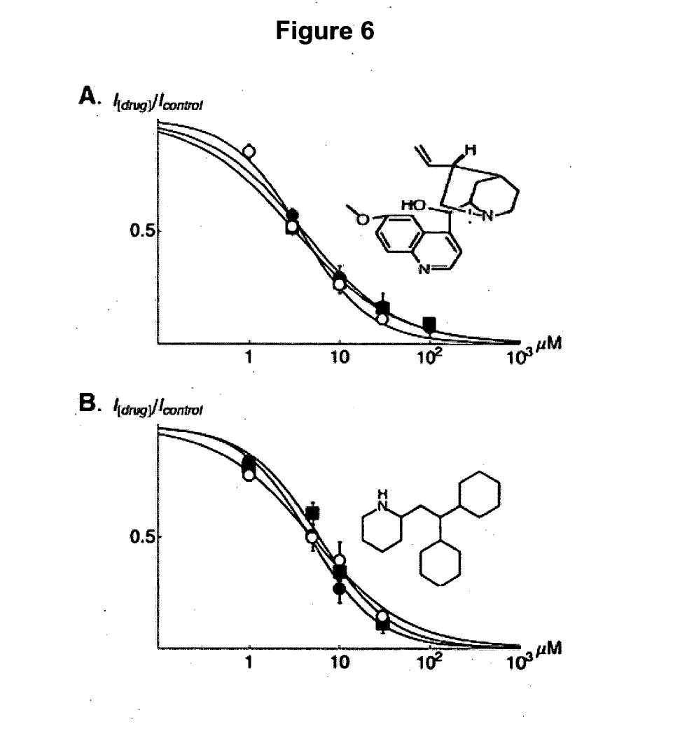

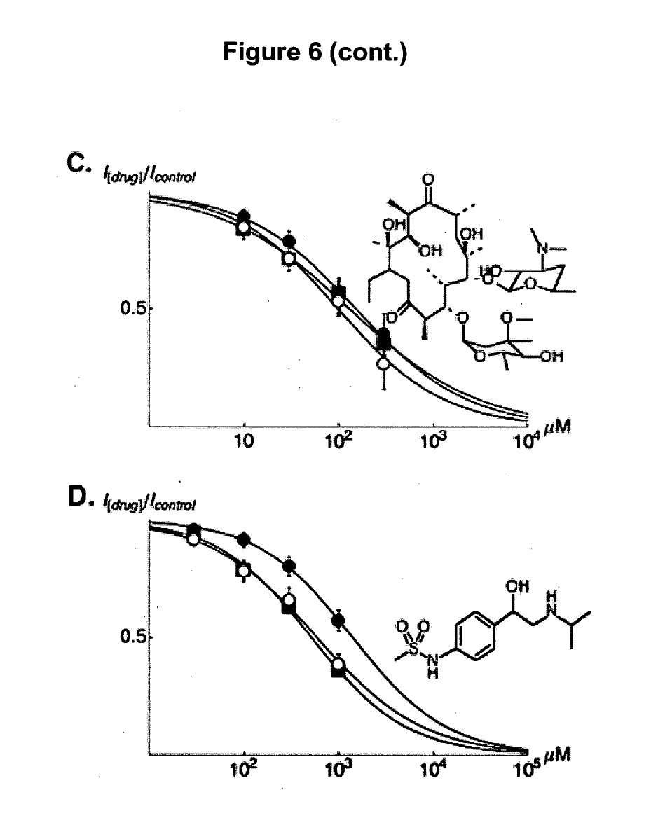

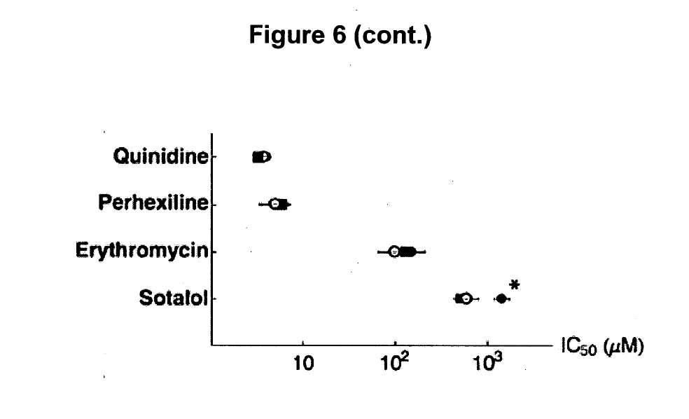

We next investigated the affinity of 4 drugs previously established to block hERG in the high nanomolar or micromolar range—quinidine, perhexiline, erythromycin, and dl-sotalol, for the N588E-hERG, WT-hERG and N588K-hERG constructs expressed in CHO cells. Typical examples of current traces recorded from WT-, N588K- and N588E-hERG in the presence and absence of 3μM quinidine are illustrated in FIG. 5. Quinidine, 3 μM, caused a similar degree of block of WT and the two N588 charge mutants. This is also seen from the summary Hill plots (FIG. 6A). Perhexiline and erythromycin showed the same pattern as that observed for quinidine, i.e. similar affinities for all hERG constructs (FIG. 6A, C). In contrast, dl-sotalol showed a significantly higher affinity for N588E-hERG and WT-hERG compared to N588K-hERG (FIG. 6D, Table 1).

### Reduced Affinity for N588K-hERG and State-Dependent Binding

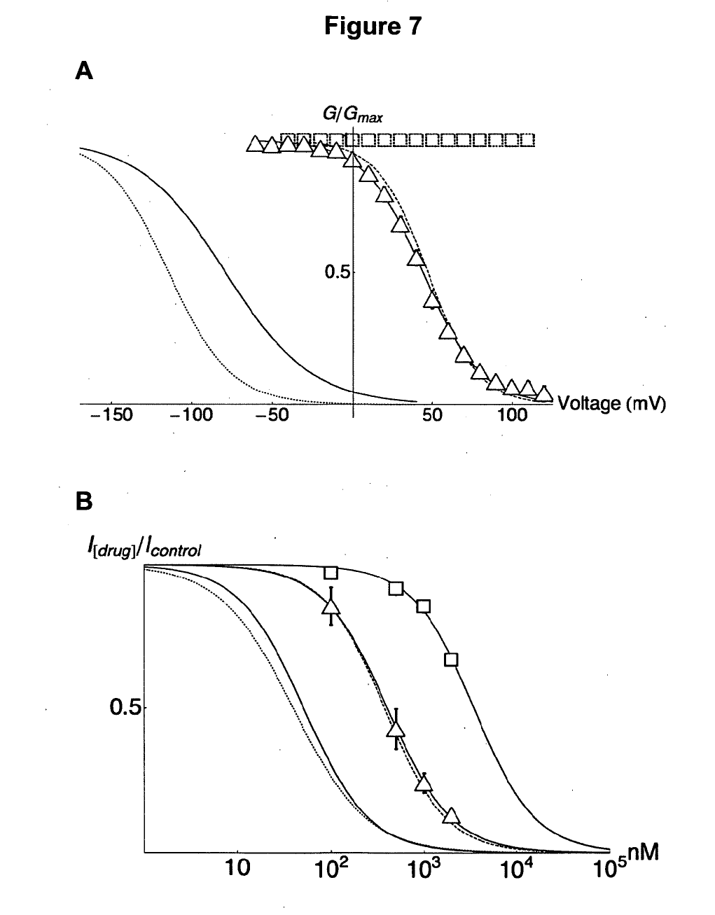

The data from FIGS. 3 and 4 clearly demonstrate that the four high-affinity drugs used in this study showed reduced affinity for the inactivation-deficient N588K-hERG channels. To determine whether this reduced affinity for N588K-hERG reflected a state-dependence of drug-binding, we investigated whether there was a similar reduced affinity for a range of inactivation-deficient mutants. Specifically, we investigated binding of dofetilide to S631A-hERG and S620T-hERG. S631A-hERG has a markedly right-shifted V_{0.5} of steady state inactivation compared to WT-hERG that is very similar to that observed for N588K (FIG. 7A) whereas S620T-hERG does not inactivate at measurable voltages. Accordingly, at +20 mV, the proportion of channels in the Open:Inactivated states is ˜95:5 for N588K and S631A, compared to 100:0 for S620T, but 5:95 for WT-hERG (FIG. 7A).

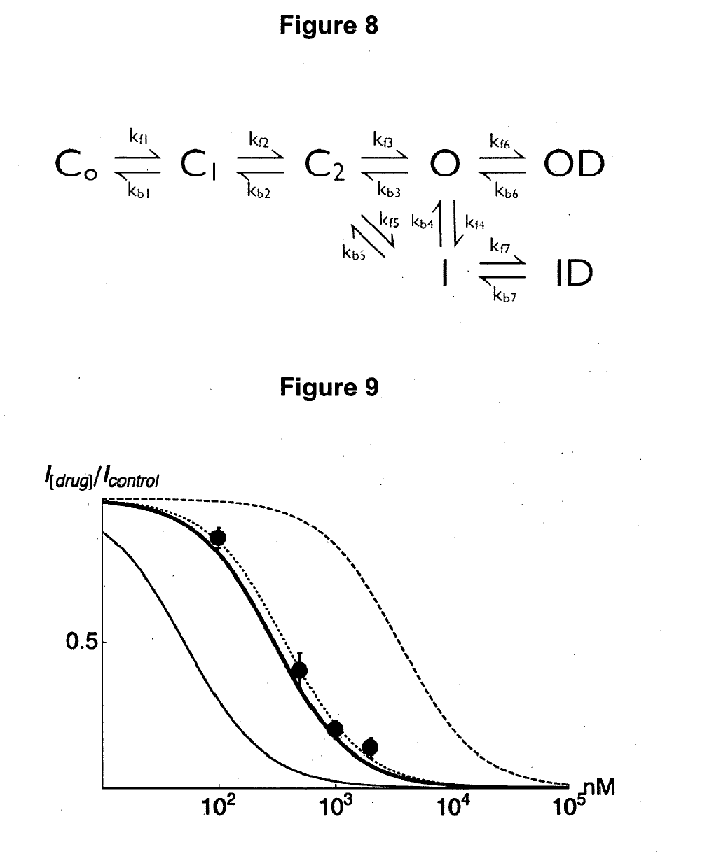

The affinity of dofetilide for S631A-hERG was not statistically different to that for N588K-hERG (404±95 nM (n=4), and 377±69 nM (n=4) respectively), an 8-fold reduction compared to WT-hERG. That these two mutants, with very similar effects on inactivation but apparently not located near each other, have very similar effects on drug binding suggests that the reduced affinity for drug binding is mediated by the reduced inactivation of the channel. However, the affinity of dofetilide for S620T, was reduced a further 10 fold (3494 nM±374 nM, n=4) (FIG. 7B) compared to its affinity for S631A or N588K. Given that there is relatively little difference in the extent to which S631A and N588K channels occupy the open state at +20 mV (the voltage at which we assayed drug binding) compared to S620T channels (i.e. ˜85% compared to 100%), a marked reduction in drug-affinity for the S620T-hERG suggests a direct effect on drug binding by this mutant. An alternative hypothesis is that, despite the relative infrequency with which S631A and N588K channels occupy the inactive state at +20 mV, the kinetics of drug binding and unbinding are such that whenever the channel enters the inactive state it binds drug that, with a very slow off rate, remains bound for a long period. According to this hypothesis, binding of drug to the S620T mutant would only encounter the open state and so reflect the affinity for the open state, whereas binding to WT or N588K channels would reflect a weighted average of the affinity for the inactive and open states dependent on the relative rates of transitions between the two states and drug binding and unbinding rates. To test this hypothesis we set up a computer model of drug binding to hERG channels as depicted in FIG. 8.

### Modelling Kinetics of Drug Binding to Open and Inactive States

The Markov chain model for hERG kinetics is based on that developed by Lu et al. (J Physiol. 2001 Dec. 15; 537(Pt 3):843-51) with the addition of two more states: drug-bound open state and drug-bound inactivated state (FIG. 8). The rate constants, scaled to 22° C., for the model are shown in the data supplement. To model the S620T mutant the rate constants for transition steps to the inactivated state were set to 0. To model the N588K mutant, the rate constants for the transition between the open and inactive states were adjusted to reproduce the experimentally observed changes in these rate constants and the steady-state inactivation. For simplicity, we assumed that the kinetics of activation were the same for WT, S620T and N588K channels.

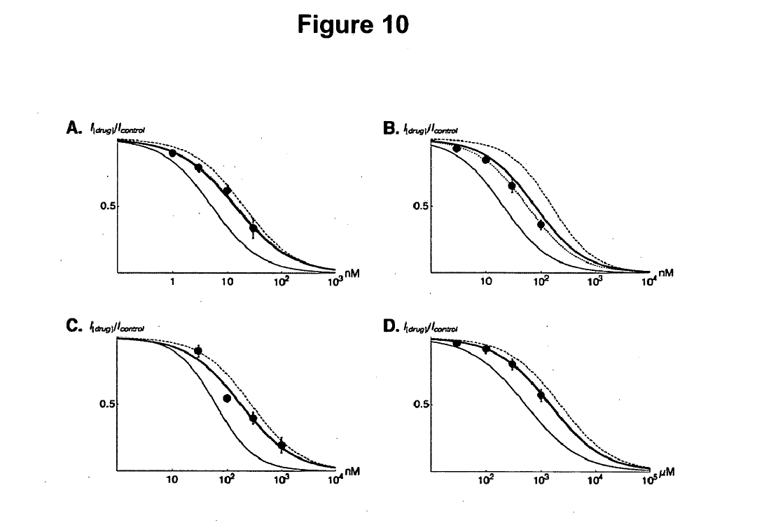

To calculate the rate constants for open and inactive state drug blocked we assumed that drug affinity for S620T represented the affinity for the open state. The rate constants for open state drug block, kf6 and kr6, accordingly were constrained to fit the data for drug binding to the S620T channels. The values for kf6 and kr6 were then held constant and the values for drug binding to the inactivated state, kf7 and kr7, constrained to fit the time course of drug block for WT channels. In the case of dofetilide binding, the calculated affinity for the open state (kf6/kr6) was 3.5 μM, i.e. the value for S620T, and the calculated affinity for the inactive state (kf7/kr7) was 47.8 nM. The measured affinity for dofetilide binding to WT channels, 50.1 nM, is much closer to the calculated value for the affinity to the inactive state reflecting both the greater proportion of time WT channels spend in the inactive state and the slower dissociation of drug from the inactive state. If the original assumption was correct, i.e. drug affinity for S620T is the true affinity of the drug for the open state, then substitution of the values for kf6, kr6, kf7 and kr7 into our model for N588K should reproduce the experimentally determined IC₅₀ for dofetilide binding to N588K. As can be seen from the data in FIG. 9, the model predicted values are very close to the experimental data. The same procedure was repeated for each of the state-dependent drugs formerly assessed (FIG. 10A-D) with similarly good approximations to the experimental data.

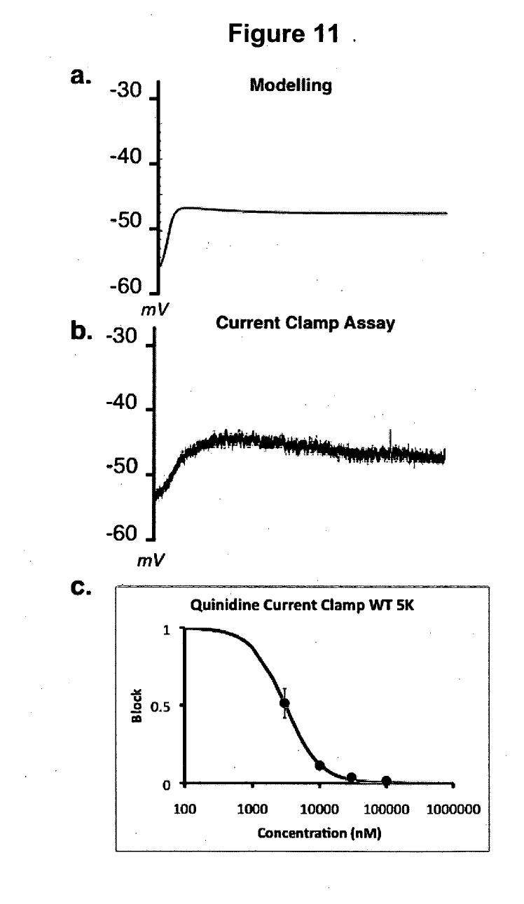

Incorporation of the model for hERG drug binding into a CHO-cell model of membrane potential was also able to reproduce the effects of drugs on membrane potential in a hERG transfected CHO-cell. In a blank CHO cell the resting membrane potential is ˜−30 mV compared to ˜−60 mV for the CHO cell line stably transfected with the hERG K+ channel. 50% block of the hERG channels causes a 3.5 mV increase in membrane potential. This agrees very well with the experimental data (FIG. 11). Furthermore, modelling studies indicate that the change in membrane potential can be enhanced from 3.5 mV to 4.5 mV per 50% reduction in hERG channel activity by shifting the voltage-dependence of activation by -15 mV.

### DISCUSSION

### State-dependence of Drug Binding

Most drugs that block hERG require channel opening. Some evidence suggests that, once open, inactivation increases drug affinity for the channel: first, mutant hERG channels with disrupted inactivation—S631A, S620T, and G628C/S631C reduce drug-block of hERG by multiple agents. Furthermore, mutations introduced into Bovine-EAG (bEAG) that enable channel inactivation also confer sensitivity to dofetilide channel block. However, the inactivation disrupting mutations employed could affect drug-block through gating independent means. S631 and S620 lie proximate to the drug-binding space, and may produce conformational changes at the base of the pore helix—an area known to be an important molecular determinant of drug-binding. G628C/S631 C markedly reduces the potassium selectivity of hERG, suggesting significant conformational changes in the vicinity of the selectivity filter have taken place in this mutant. Further, the mutant hERG channels G648A and S623A both promote inactivation, but reduce methanesulfonamide block of hERG. Adding to the difficulty of interpreting whether drug binding is state-dependent, voltage protocols designed to favour occupancy of the inactivated state during drug-binding (i.e. large depolarisations) also relieve drug block.

The data reported in this application provides the strongest evidence to date that drug binding to hERG K+ channels can be state dependent. Firstly, we have shown that a mutation introduced into a region thought to be remote from the drug binding pocket yet still affects inactivation (N588K) affects drug binding. In the absence of a crystal structure of hERG in open and inactive states we cannot be certain that the position of N588K is remote from the drug binding pocket, however a combination of structural studies, toxin binding studies and molecular dynamics modelling studies suggest that N588 on the hydrophilic surface of the S5P α-helix will be facing the extracellular space and therefore well away from the drug binding pocket. Secondly, we have shown that two distinct mutants that have very similar effects on inactivation (N588K and S631A) have the same effect on drug binding. The simplest explanation for this observation is that it is the effect these mutations have on inactivation that accounts for the altered drug binding. Thirdly, our kinetic modelling of drug binding to WT, S620T and N588K mutant channels shows that all the differences in drug binding between these mutants can be explained simply on the basis of differences in occupancy of open and inactive states and the kinetics of drug binding and unbinding. An important corollary of our kinetic modelling studies is that for the first time we have been able to estimate the affinities for both the open and inactive states for a range of drugs. Furthermore, our data suggest that the affinities for the open and inactive states of any drug can now be calculated if one knows the affinity for WT and S620T mutant channels.

In contrast with our data, there is one report in the literature that suggests that the S620T mutation could have a direct effect on drug binding. It was shown that S620T and S620C, whilst having the same effects on inactivation gating (abolition), had different affinities for E-4031—a methanesulfonamide similar to dofetilide. This was interpreted as indicating that the Serine side chain at 620 contributed directly to drug binding. This data, however, is not necessarily incompatible with our data. It is possible that the abolition of inactivation alters drug affinity in both the S620T and S620C but that the cysteine side chain in S620C, but not the threonine side chain in S620T, can bind to the drug in the open state and thereby increase the affinity relative to that observed for S620T. Further, drug block in their studies was assessed after a 4s depolarising step to +50 mV. At this voltage the ◯:I probability for S631A should be ˜1:1, significantly different to S620C which does not inactivate. However, the affinity of E-4031 for S631A-and S620C-hERG was similar suggesting that one or the other alters drug block with some independence from gating mechanisms. Our data for dofetilide block of S631A and N588K, i.e. S631A affects drug block by altered gating, implies that it is S620C which exerts a direct effect on drug binding.

Relevance for High Throughput Screens for Drug Binding to hERG

Given the mandated need to screen all drugs for hERG binding, there has been considerable effort put into developing high throughput screens for assaying drug binding to hERG. Such assays have included fluorescence based voltage assays and Rb+ efflux assays. In general, however, the results of these screens have been poor and we suggest that this is because they predominantly assay binding to the open state and therefore fail to detect many if not all high affinity blockers. Our data show that the difference in affinity between the open and inactive states can be 70-fold. Therefore, it is imperative that to be successful any high throughput screening system adequately assay binding to the inactive state.

Our modelling studies also indicate that mutations that result in a leftward shift in the voltage-dependence of activation will increase the voltages shifts caused by drug block of hERG channels and thereby facilitate high throughput drug screening assays based on measuring changes in voltage.

### OBSERVATIONS

We investigated the relationship between inactivation gating and drug-block of hERG, finding that high affinity block (nanomolar range) is promoted by inactivation. The use of charged mutants at N588 provides a methodology for investigating the conformational changes of the channel pore between open and inactivated states. Further, we have calculated for the first time the relative affinities of drug binding to the open and inactive states of the hERG channel, which in the case of dofetilide shows a 70-fold greater affinity for the inactive state. The pharmaceutical importance of these data is highlighted by the observation that two drugs that have been withdrawn from the market; astemizole and terfenadine and one that has had its use severely restricted; cisapride) exhibit a marked preference for binding to the inactive state.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An isolated nucleic acid molecule capable of expressing an human ether-à-go-go related gene (hERG) mutant that enhances hERG inactivation, wherein the mutation is not located in the vicinity of the drug-binding pocket within the cavity of the ion conduction pore of the hERG protein.

2. The nucleic acid molecule according to claim 1 wherein the mutation is located on the alpha-helix of the S5P linker.

3. The nucleic acid molecule according to claim 2 wherein the mutation is N588E, H587K, G584S, or Q592D.

4. The nucleic acid molecule according to claim 3 wherein the mutation is N588E.

5. The nucleic acid molecule according to claim 1 containing one or more mutations as substantially as set out in FIG. 12 (SEQ ID NO: 1).

6. The nucleic acid molecule according to any one of claims 1 to 4 further containing one or more mutations selected from L524W, L532P, V535A, A536V or K538Q that promotes activation of the hERG ion channel.

7. A vector for expressing an isolated nucleic acid molecule according to any one of claims 1 to 6.

8. The vector according to claim 7 containing a promoter suitable for expression in a mammalian cell and an antibiotic resistance gene to aid selection of cells expressing the hERG channel.

9. A stable cell line expressing an isolated nucleic acid molecule according to any one of claims 1 to 6.

10. A stable cell line containing a vector according to claim 7 or 8 and expressing a hERG mutant protein.

11. The stable cell line according to claim 8 or 9 selected from Chinese hamster ovary cell, human embryonic kidney cell or transformed African Green Monkey kidney fibroblast cell.

12. An assay for screening potential drug binding to hERG protein comprising:
a stable cell line according to any one of claims 9 to 11; and
means to determine activity of the hERG protein in the cell.

13. The assay according to claim 12 involving either ion flux or voltage changes then using either patch clamp assays, Rb+ efflux assays as a surrogate for K+ flux or fluorescence to assay voltage changes of the cell.

14. The assay according to claim 12 or 13 wherein a test drug is incubated with the cells and the effects on ion flux or voltage is monitored.

15. Use of the stable cell line according to any one of claims 9 to 11 in an assay for screening potential drug binding to hERG protein.

16. Use of the isolated nucleic acid molecule according to any one of claims 1 to 6 an assay for screening potential drug binding to hERG protein.


SEQUENCE LISTING (10 sequences)

SEQ ID NO: 1 - DNA, 3486 bp, human
  misc_feature (1572)..(1572): n is a, c, g, or t
atgccggtgcggaggggccacgtcgcgccgcagaacaccttcctggacaccatcatccgcaagtttgagggccagagccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatgggagctgcttcctatgtctggtggatgtggtgcccgtgaagaacgaggatggggctgtcatcatgttcatcctcaatttcgaagtggtgatggagaaggacatggtggggtccccggctcatgacaccaaccaccggggcccccccaccagctggctggccccaggccgcgccaagaccttccgcctgaagctgcccgcgctgctggcgctgacggcccgggagtcgtcggtgcggtcgggcggcgcgggcggcgcgggcgccccgggggccgtggtggtggacgtggacctgacgcccgcggcacccagcagcgagtcgctggccctggacgaagtgacagccatggacaaccacgtggcagggctcgggcccgcggaggagcggcgtgcgctggtgggtcccggctctccgccccgctcagcgcccggccagctcccatcgccccgggcgcacagcctcaaccccgacgcctcgggctccagctgcagcctggcccggacgcgctcccgagaaagctgcgccagcgtgcgccgcgcctcgtcggccgacgacatcgaggccatgcgcgccggggtgctgcccccgccaccgcgccacgccagcaccggggccatgcacccactgcgcagcggcttgctcaactccacctcggactccgacctcgtgcgctaccgcaccattagcaagattccccaaatcaccctcaactttgtggacctcaagggcgaccccttcttggcttcgcccaccagtgaccgtgagatcatagcacctaagataaaggagcgaacccacaatgtcactgagaaggtcacccaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgcttaaggagacggaagaaggcccgccggcgaccgagtgtggctatgcatgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgyknaagacggcgcgcctgctgcggyyngtgcgcgyngyncggmarctggatcgctactcagagtacggcgcggccgtgctgttcttgctcatgtgtacctttgcgctcatcgcgcactggctagcctgcatctggtacgcaattggcaacatggagcagccacacatggactcacgcatcdsntggctgmanrayctgggcgaccagataggcaaaccctacaacagctcaggcctgggcggcccctccatcaaggacaagtatgtgacggcgctctacttcaccttcagcagcctcaccagtgtgggcttcggcaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccggtcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctggggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctctgatcacttctggtccagcctggagatcaccttcaacctgcgagataccaacatgatcccgggctcccccggcagtacggagttagagggtggcttcagtcggcaacgcaagcgcaagttgtccttccgcaggcgcacggacaaggacacggagcagccaggggaggtgtcggccttggggccgggccgggcgggggcagggccgagtagccggggccggccgggggggccgtggggggagagcccgtccagtggcccctccagccctgagagcagtgaggatgagggcccaggccgcagctccagccccctccgcctggtgcccttctccagccccaggccccccggagagccgccgggtggggagcccctgatggaggactgcgagaagagcagcgacacttgcaaccccctgtcaggcgccttctcaggagtgtccaacattttcagcttctggggggacagtcggggccgccagtaccaggagctccctcgatgccccgcccccacccccagcctcctcaacatccccctctccagcccgggtcggcggccccggggcgacgtggagagcaggctggatgccctccagcgccagctcaacaggctggagacccggctgagtgcagacatggccactgtcctgcagctgctacagaggcagatgacgctggtcccgcccgcctacagtgctgtgaccaccccggggcctggccccacttccacatccccgctgttgcccgtcagccccctccccaccctcaccttggactcgctttctcaggtttcccagttcatggcgtgtgaggagctgcccccgggggccccagagcttccccaagaaggccccacacgacgcctctccctaccgggccagctgggggccctcacctcccagcccctgcacagacacggctcggacccgggcagtacgcgttag

SEQ ID NO: 2 - DNA, 21 bp, human
cttctcctactgttattgtgg

SEQ ID NO: 3 - DNA, 30 bp, human
cttctcctactgttattgcctcccccaccg

SEQ ID NO: 4 - DNA, 24 bp, human
gttgtcgtagtggctgccgcagcg

SEQ ID NO: 5 - DNA, 24 bp, human
gttgtcgtagtggctgccgcagcg

SEQ ID NO: 6 - DNA, 12 bp, human
aaaaagcaacag

SEQ ID NO: 7 - DNA, 30 bp, human
ggtggcggagggtcttcctcatcgagtagc

SEQ ID NO: 8 - DNA, 12 bp, human
catcacaaaaag

SEQ ID NO: 9 - DNA, 12 bp, human
aataacgatgac

SEQ ID NO: 10 - DNA, 3480 bp, human
atgccggtgcggaggggccacgtcgcgccgcagaacaccttcctggacaccatcatccgcaagtttgagggccagagccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatgggagctgcttcctatgtctggtggatgtggtgcccgtgaagaacgaggatggggctgtcatcatgttcatcctcaatttcgaggtggtgatggagaaggacatggtggggtccccggctcatgacaccaaccaccggggcccccccaccagctggctggccccaggccgcgccaagaccttccgcctgaagctgcccgcgctgctggcgctgacggcccgggagtcgtcggtgcggtcgggcggcgcgggcggcgcgggcgccccgggggccgtggtggtggacgtggacctgacgcccgcggcacccagcagcgagtcgctggccctggacgaagtgacagccatggacaaccacgtggcagggctcgggcccgcggaggagcggcgtgcgctggtgggtcccggctctccgccccgcagcgcgcccggccagctcccatcgccccgggcgcacagcctcaaccccgacgcctcgggctccagctgcagcctggcccggacgcgctcccgagaaagctgcgccagcgtgcgccgcgcctcgtcggccgacgacatcgaggccatgcgcgccggggtgctgcccccgccaccgcgccacgccagcaccggggccatgcacccactgcgcagcggcttgctcaactccacctcggactccgacctcgtgcgctaccgcaccattagcaagattccccaaatcaccctcaactttgtggacctcaagggcgaccccttcttggcttcgcccaccagtgaccgtgagatcatagcacctaagataaaggagcgaacccacaatgtcactgagaaggtcacccaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttcggcaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctggggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctccgaccacttctggtccagcctggagatcaccttcaacctgcgagataccaacatgatcccgggctcccccggcagtacggagttagagggtggcttcagtcggcaacgcaagcgcaagttgtccttccgcaggcgcacggacaaggacacggagcagccaggggaggtgtcggccttggggccgggccgggcgggggcagggccgagtagccggggccggccgggggggccgtggggggagagcccgtccagtggcccctccagccctgagagcagtgaggatgagggcccaggccgcagctccagccccctccgcctggtgcccttctccagccccaggccccccggagagccgccgggtggggagcccctgatggaggactgcgagaagagcagcgacacttgcaaccccctgtcaggcgccttctcaggagtgtccaacattttcagcttctggggggacagtcggggccgccagtaccaggagctccctcgatgccccgcccccacccccagcctcctcaacatccccctctccagcccgggtcggcggccccggggcgacgtggagagcaggctggatgccctccagcgccagctcaacaggctggagacccggctgagtgcagacatggccactgtcctgcagctgctacagaggcagatgacgctggtcccgcccgcctacagtgctgtgaccaccccggggcctggccccacttccacatccccgctgttgcccgtcagccccctccccaccctcaccttggactcgctttctcaggtttcccagttcatggcgtgtgaggagctgcccccgggggccccagagcttccccaagaaggccccacacgacgcctctccctaccgggccagctgggggccctcacctcccagcccctgcacagacacggctcggacccgggcagttag